(19) United States
(12) Patent Application Publication
(10) Pub. No.: US 2024/0009404 A1
(43) Pub. Date: Jan. 11, 2024

(54) DEVICES AND METHODS FOR SHARPS DISPOSAL

(71) Applicant: BLEXX TECHNOLOGY, LLC, Roseland, IN (US)

(72) Inventors: ERIN Elizabeth Christiansen, Roseland, IN (US); RAY MCMULLEN, Shorewood, WI (US); FERNANDO BUARQUE, White Bear Lake, MN (US); GARY HASSEBROEK, Coon Rapids, MN (US); SCOTT KALPIN, Rush City, MN (US); BRAD MILOS, Ramsey, MN (US); GREG BROGE, Arden Hills, MN (US)

(21) Appl. No.: 18/038,478

(22) PCT Filed: Dec. 27, 2021
(86) PCT No.: PCT/US21/65217

§ 371 Date: May 24, 2023

Related U.S. Application Data
(60) Provisional application No. 63/131,926, filed on Dec. 30, 2020.

(51) Int. Cl. A61M 5/32

(52) U.S. Cl. CPC A61M 5/3278; A61M 2005/3282; A61B 2017/00398

(57) ABSTRACT

Provided herein are devices, systems, and methods for disposal of medical sharps. In particular, provided herein are devices, systems, and methods for safe and efficient destruction of medical and research sharps (e.g., needles).

### FIELD

Provided herein are devices, systems, and methods for disposal of medical sharps. In particular, provided herein are devices, systems, and methods for safe and efficient destruction of medical and research sharps (e.g., needles).

### BACKGROUND

Health care workers, lab workers, sanitation workings, and housekeeping staff are at substantial risk of acquiring bloodborne pathogen infections through exposure to blood or infectious body fluids. Common pathogens obtained from needle sticks include hepatitis B virus, Hepatitis C virus, and human immunodeficiency virus.

One recent study found that 35.6% of Egyptian health workers were exposed to at least 1 needlestick injury during the past 3 months with an estimated annual number of 4.9 needlesticks per worker (Talaat et al, AJIC, 31: 469). Further studies revealed high levels of needle sticks and unsafe handling of needles among health care workers in India (Muralidhar et al., Indian J Med Res 131, March 2010, pp 405-410), the UK (Thomas and Murray, Ann R Coll Surg Engl 2009; 91: 12-17), and Malaysia (Norsayani and Hassim, Ann R Coll Surg Engl 2009; 91: 12-17). In addition, sanitation workers and housekeeping workers are at risk during disposal of needles in household and other trash collection (e.g., from home or recreational users of needles).

Many needlestick injuries occur during disposal of needles by users that do not follow appropriate disposal protocols (e.g., re-capping needles, failing to use a proper disposal container, or misuse of disposal containers). While this problem has been prevalent for many, many, years, it remains unsolved. Improved disposal systems and methods are needed in order to reduce injuries and infection.

### SUMMARY

Provided herein are devices, systems, and methods for disposal of medical sharps. In particular, provided herein are devices, systems, and methods for safe and efficient destruction of medical and research sharps (e.g., needles).

The devices and methods described herein solve the problem of safe and efficient sharps disposal by providing a small, inexpensive, and convenient method of safely disposing of sharps (e.g., needles) at the location of use. This eliminates risk of pathogen exposure for both medical workers and waste disposal workers. The present disclosure is exemplified with the disposal of needles. However, the present disclosure is not limited to the disposal of needles. Any medical, research, or other sharp may be disposed of using the compositions and methods described herein.

For example, in some embodiments, provided herein is a sharps (e.g., needle) disposal device, comprising: a) a needle entry port operably linked to a rotating carousel; b) a destruction component comprising a blade configured to chop the needle to debris of a predetermined debris size (e.g., less than 5, 4, 3, or 2 mm in length); and c) a disposal canister configured to collect the debris. In some embodiments, the blade comprises an oscillating component and a stationary component.

In some embodiments, the canister is operably linked to the destruction component via a needle chamber and a plunger, wherein the plunger is configured to advance the needle into the destruction component. In some embodiments, the rotating carousel comprises a plurality of compartments, wherein each of the compartments is configured to hold a single needle. In some embodiments, the needle entry port comprises a detector that detects entry of the needle into the port. In some embodiments, the detector counts the number of needles inserted in the needle entry port over time. In some embodiments, the device further comprises an internal power source.

In some embodiments, the destruction component and the disposal canister are contained in a replaceable cartridge. In some embodiments, the replaceable cartridge is disposable. In some embodiments, the replaceable cartridge is sealed. In some embodiments, the replaceable cartridge is connected to the carousel via a canister mount. In some embodiments, the replaceable cartridge further comprises a battery. In some embodiments, the device further comprises an indicator (e.g., indicator light) that indicates when the disposal canister is full.

In some embodiments, the device further comprises a drive motor and gear box component configured to drive the blade. In some embodiments, the blade is configured to move in a reciprocating manner. In certain embodiments, the drive motor is configured to start moving the blade when a needle contacts the destruction module and stop moving when the needle or needle/syringe is destroyed.

The present disclosure is not limited to a particular needle or sharp. Examples include but are not limited to, scalpel blades, hypodermic needles with or without syringes, suture needles.

Further embodiments provide a method of disposing a needle, comprising: a) inserting a needle in the needle entry port of a device described herein; b) advancing the needle to the destruction component; and c) destroying the needle using the destruction component. In some embodiments, the method further comprises the step of removing the replaceable cartridge and installing a new replaceable cartridge. In some embodiments, the device is used in a home, a hospital, a nursing home, a public restroom, a research lab, a hotel, a clinic, or an ambulance.

Additional embodiments are described herein.

### BRIEF DESCRIPTION OF THE DRAWINGS

FIG. 1 shows an overview of an exemplary device of embodiments of the present disclosure.

FIG. 2 shows a needle entry port of an exemplary device of embodiments of the present disclosure.

FIG. 3 shows a needle carousel of an exemplary device of embodiments of the present disclosure.

FIG. 4 shows a needle carousel of an exemplary device of embodiments of the present disclosure.

FIG. 5 shows a needle carousel and needle destruction module of an exemplary device of embodiments of the present disclosure.

FIG. 6 shows a needle destruction module of an exemplary device of embodiments of the present disclosure.

FIG. 7 shows a replaceable cartridge of an exemplary device of embodiments of the present disclosure.

FIG. 8 shows a replaceable cartridge of an exemplary device of embodiments of the present disclosure.

FIG. 9 shows a cut-out view of a replaceable cartridge of an exemplary device of embodiments of the present disclosure.

### DETAILED DESCRIPTION

Provided herein are devices, systems, and methods for disposal of medical sharps. In particular, provided herein are devices, systems, and methods for safe and efficient destruction of medical and research sharps (e.g., needles).

The most prevalent solution for hypodermic needle and syringe or other sharps disposal is a sharps disposal container offered by medical waste disposal companies. They are installed in many public places such as lavatories, airplanes and other public and medical facilities for convenient needle/syringe disposal. These containers are typically exchanged either through a mail-in program where the boxes are packaged and shipped by the customer or a full-service program where the containers are consolidated and transported by a medical waste disposal company. The additional costs and time required for disposal makes sharps disposal containers inconvenient and out of reach for some users.

Accordingly, provided herein is a low-cost solution for disposal of needles and syringes that safely destroys needles and syringes without adding additional disposal cost or time. The devices described herein utilize a self-contained, removable, and disposable cartridge that sequesters needle debris. Disposal does not require contact with a blade or needle debris.

In the present disclosure and claims, the terms “needle” and “needle and syringe” are used interchangeably to refer to a needle, with or without an attached syringe. The devices described herein are capable of destroying and disposing of needles with or without attached syringes.

In the present disclosure, the devices and methods are exemplified with needles comprising syringes (e.g., hypodermic needles). However, the devices described herein are suitable with use for other sharps (e.g., including but not limited to, suture needles and scalpel blades).

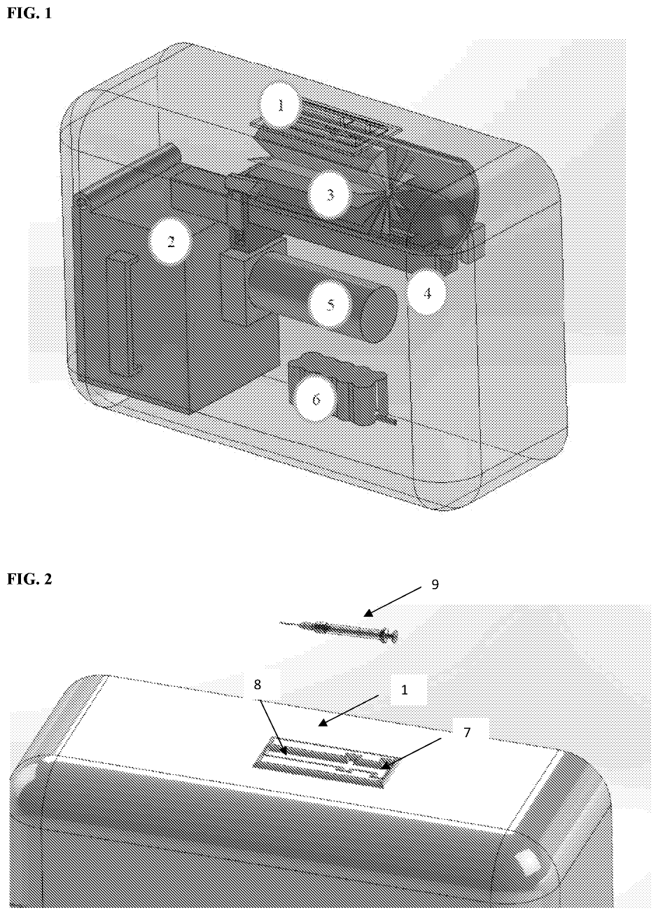

Exemplary devices are described in FIGS. 1-9.

FIG. 1 shows an exemplary needle disposal device of the present disclosure. Shown are entry port 1, replaceable cartridge 2, carousel 3, chamber/plunger component 4, drive motor/gear box 5, and internal power source 6 (e.g., lithium battery pack) to provide power to the device. Each of these components is discussed in detail below.

FIG. 2 shows a detailed view of entry port 1. The entry port 1 controls what can be inserted into the device. In some embodiments, as shown in FIG. 2, a needle 9 (shown in FIG. 2 with an integrated syringe) is placed in the entry receptacle 7 of the entry port 1. In some embodiments, the geometry of entry receptacle 7 is in a needle/syringe shape as shown in FIG. 2, although other geometries are specifically contemplated for other sharps.

Still referring to FIG. 2, after a needle 9 is placed in the entry receptacle 7, the needle drops through opening 8 into the interior of the device. In some embodiments (e.g., as shown in FIG. 2), opening 8 is completely or partially open to the interior of the device. In some embodiments, opening 8 comprises a full or partial seal or door. In some embodiments, the pressure of the needle on the opening 8 releases the seal or door allowing the needle 9 to drop into the interior of the device.

In some embodiments, the entry port 1 comprises a detector or sensor that detects the presence of a needle and/or counts the number of needles places in the entry port 1. In some embodiments, the detector also releases a door or seal (if present), allowing the needle to drop into the interior of the device.

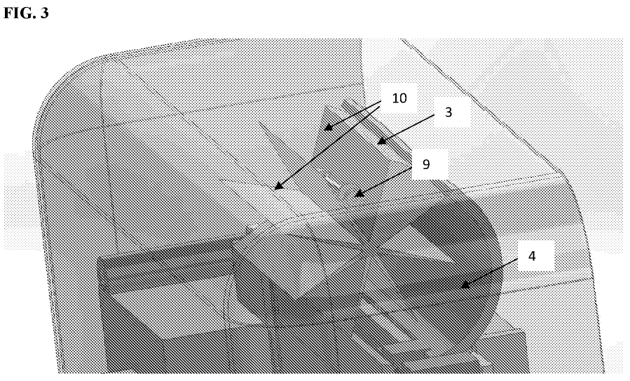

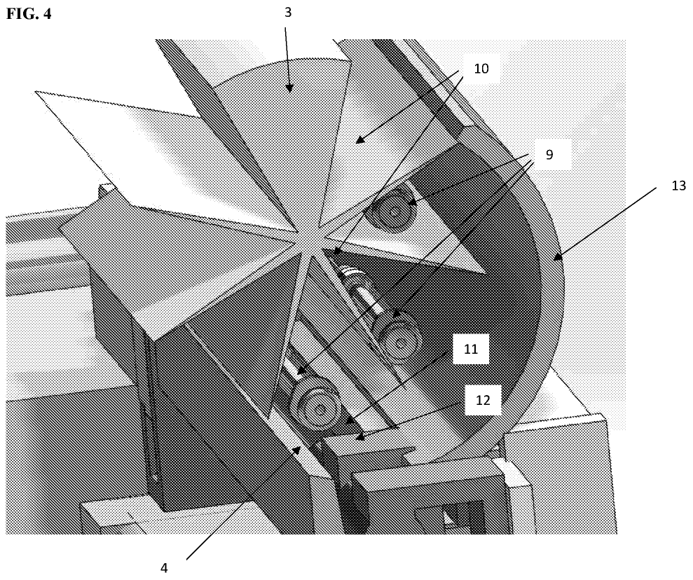

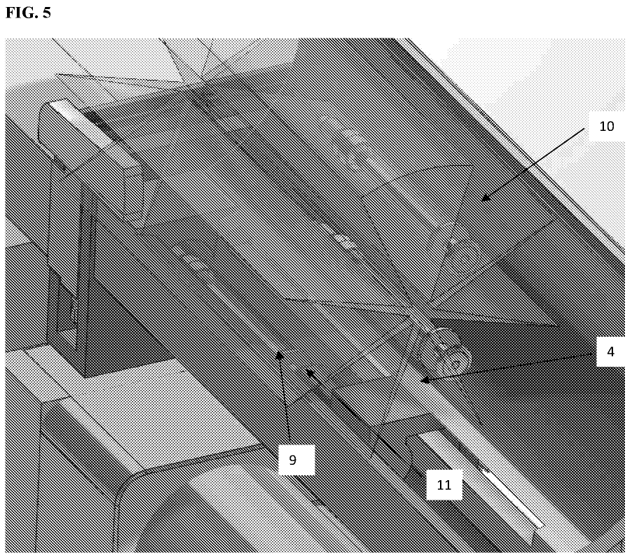

Now referring to FIG. 3, shown is carousel 3. Carousel 3 queues needles 9 that are placed in entry port 1 and deposits them into chamber/plunger component 4. After the needle drops through opening 8 of entry port 1 (not shown in FIG. 3), the needle enters a compartment 10 of carousel 3. In FIG. 3, carousel 3 is shown as a wheel configuration, although other geometries of carousel 3 and compartments 10 are specifically contemplated.

Now referring to FIG. 4, shown is a close up of carousel 3. As shown, each compartment 10 of carousel 3 is able to hold one or more needles 9. Multiple needles 9 can be simultaneously placed in carousel 3 (e.g., with a single needle 9 is each compartment 10 as shown in FIG. 4 or in other configurations). In some embodiments, after a needle drops into a compartment 10, the carousel 3 rotates (e.g., either clockwise or counterclockwise), exposing an empty compartment 10 for the next needle 9.

Still referring to FIG. 4, also shown are details of the chamber/plunger component 4. Shown are chamber 11, plunger 12, and housing 13. The chamber/plunger component 4 feeds needles/syringes into replaceable cartridge 2 for destruction. As described in more detail below, carousel 3 rotates, allowing a needle 9 to be deposited into chamber/plunger component 4.

Now referring to FIG. 5, shown is a close-up view of needle 9 in the chamber 11 of chamber/plunger component 4. As the carousel 3 rotates, a single needle 9 drops into the chamber 11. In the view shown in FIG. 4, each chamber/plunger component 4 has a single chamber 11 and plunger 12, although other configurations are specifically contemplated.

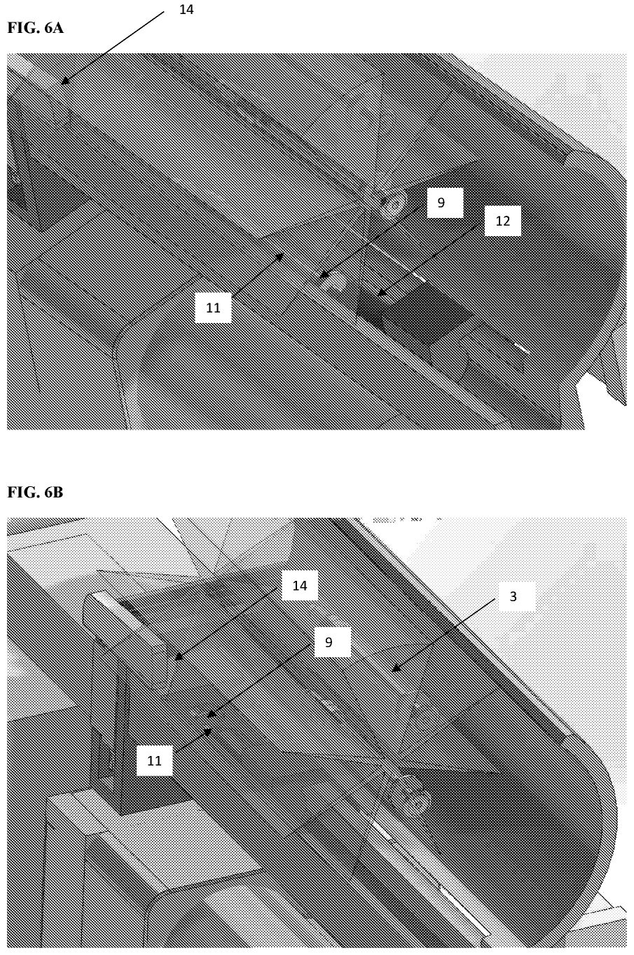

Now referring to FIG. 6, shown is advancement of needle 9 into destruction module 14. In FIG. 6A, a needle 9 in chamber 11 is advanced by the action of plunger 12 towards destruction module 14. FIG. 6B shows a needle 9 that has been advanced into destruction module 14. In some embodiments, as the plunger 12 advances a needle 9 towards the destruction module 14, a blade contained in the destruction module begins to cycle and chop the needle to a predetermined debris size (e.g., less than 5, 4, 3, or 2 mm in length). In some embodiments, the blade of the destruction module 14 is positioned such that the debris falls into the disposal canister 16 located in the replaceable cartridge 2 (not shown in FIG. 6). After a needle has entered the destruction module, carousel 3 advances to allow the next needle to drop into chamber 11 and the process is repeated.

In some embodiments, the blade of the destruction module is connected to a reciprocating geared drive motor system by virtue of a quick release mechanism. When the needle is completely destroyed, the drive motor shuts off. In some embodiments, the system counts the number of destructions and indicates to the operator (e.g., via an indicator light) when the disposal canister is full and replacement of the replaceable cartridge is required.

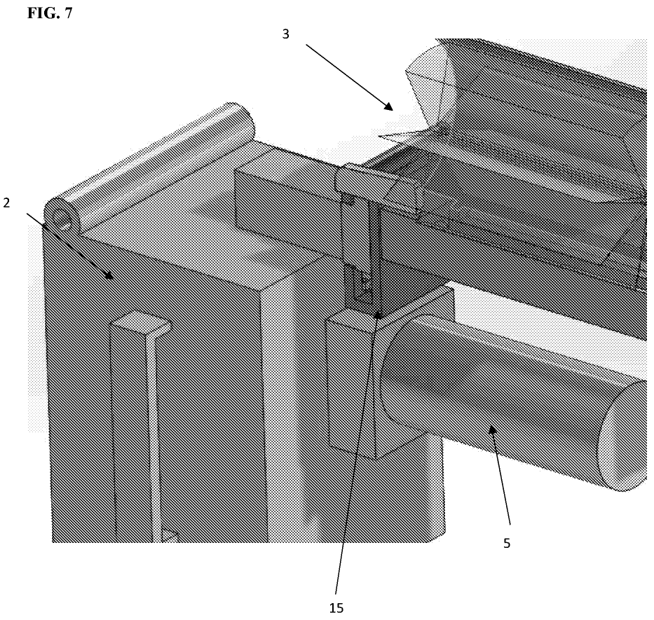

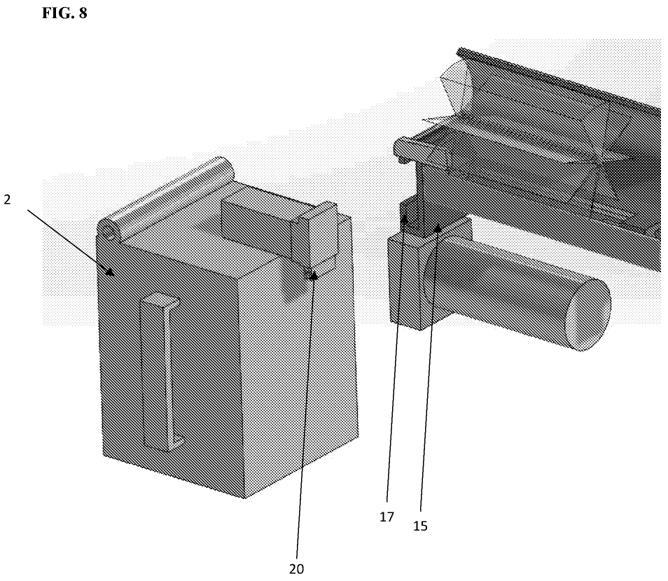

FIG. 7 shows an overview of internal device components. Shown is replaceable cartridge 2, canister mount 15, carousel 3, and drive motor and gear box 5. Replaceable cartridge 2 includes disposal canister 16, destruction module 14, and a power source (e.g., alkaline battery) to recharge the internal power (not shown in FIG. 7). The drive motor and gear box 5 converts circular motion into reciprocation motion for the destruction blade, which is located in the replaceable cartridge 2.

FIG. 8 illustrates replacement of replaceable cartridge 2. The cartridge is removed by sliding the cannister mount slider 20 of the cartridge 2 from the guides 17 of canister mount 15. A new cartridge is inserted by placing on guides 17 and securing.

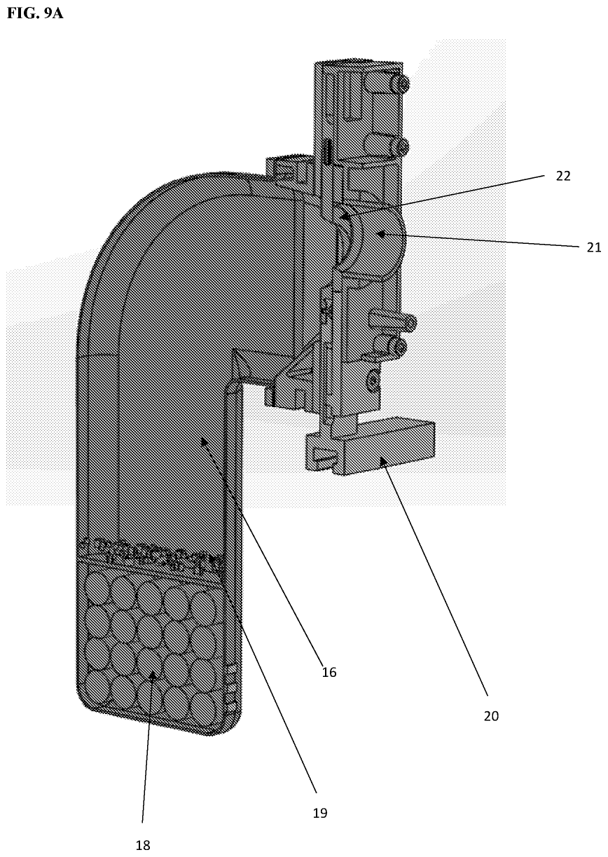

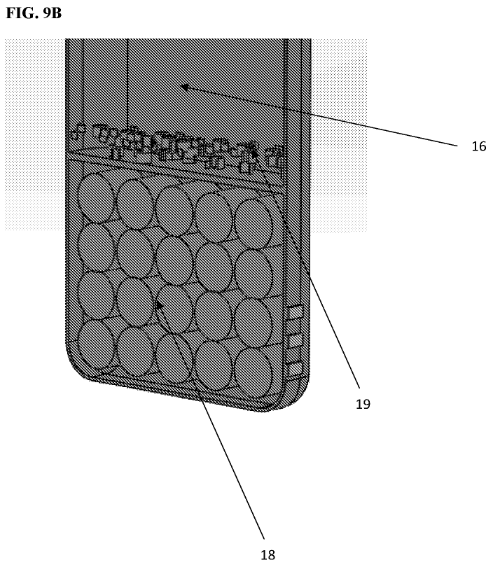

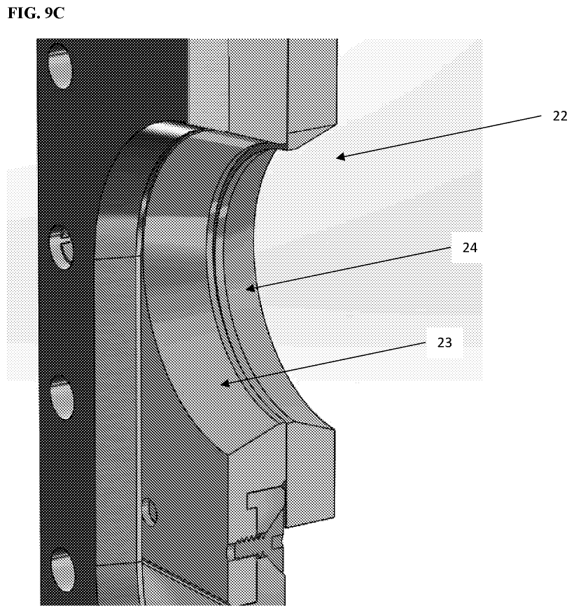

FIG. 9 shows a cut-out view of replaceable cartridge 2. FIG. 9A shows an overview of the cartridge. Shown is cannister mount slider 20, needle opening 21, disposal cannister 16, needle debris 19, blade/cutter assembly 22, and power source 18. The needle opening 21 allow a needle 9 (not shown in FIG. 9A) to be pushed from chamber 11 to blade assembly 22.

Now referring to FIG. 9B, shown is a close-up view of power source 18 (e.g., battery pack), and disposal cannister 16 comprising needle/syringe debris 19. The present disclosure is not limited to the shape or size of disposal cannister 16 shown in FIG. 9B. Other configurations are specifically contemplated.

Now referring to FIG. 9C, shown is a close-up view of blade assembly 22. Shown is oscillating component 23 and stationary component 24. The blade/cutter operates through reciprocal action of oscillating component 23 against stationary component 24. The blade assembly shown in FIG. 9C is exemplary. Additional cutting mechanisms and blades are specifically contemplated.

The present disclosure is not limited to a particular capacity of the disposal canister. In some embodiments, the canister is sized to hold 300 syringes with needles before disposal is required, although the amount will vary based on the size of the needles and syringes. Disposal canisters of different sizes are specifically contemplated (e.g., for use in different settings or with different sizes of needles and syringes).

In some embodiments, the device is configured for mounting on or near a garbage can or other waste disposal device. In some such embodiments, the device of the present invention comprises a mounting clip that permits the device to mount over a top edge of a garbage can.

In use, a user uncaps a needle and uses the needle as indicated (e.g., to inject a human or non-human subject). The present disclosure is not limited to particular needle types. Examples include, but are not limited to, medical needles with attached syringe, IV needles, blood collection needles, suture needles, etc. In some embodiments, the needle comprises a plastic or glass spacer that attaches the needle to the syringe. In some embodiments, the needle is any gauge commonly used in research or medicine (e.g., 14, 15, 16, 17, 18, 19, 20, 21, 22, 23, 25, 25, 26, 27, 27, 29, or 30 gauge). The user then inserts the needle (e.g., with or without syringe) into the entry port of a device described herein. The needle is destroyed and debris is contained in the cartridge. When the cartridge is full (e.g., as indicated by an indicator light or other alert), the user removes the replaceable cartridge and inserts a new cartridge. The full cartridge is placed in a standard waste receptacle.

The devices described herein find use in a variety of public and medical settings (e.g., including, but not limited to, a home, a hospital, a nursing home, a public restroom, a research lab, a hotel, a clinic, or an ambulance).

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

What is claimed is:

1. A needle disposal device, comprising:
a) a needle entry port operably linked to a rotating carousel;
b) a destruction component comprising a blade configured to chop said needle to a predetermined debris size; and
c) a disposal canister configured to collect said debris.

2. The device of claim 1, wherein said canister is operably linked to said destruction component via a needle chamber and a plunger, wherein said plunger is configured to advance said needle into said destruction component.

3. The device of claim 1 or 2, wherein said rotating carousel comprises a plurality of compartments, wherein each of said compartments is configured to hold a single needle.

4. The device of any of the preceding claims, wherein said needle entry port comprises a detector that detects entry of said needle into said port.

5. The device of claim 4, wherein said detector counts said needles.

6. The device of any of the preceding claims, wherein predetermined debris size is no more than approximately 3 mm in length.

7. The device of any of the preceding claims, wherein said destruction component and said disposal canister are contained in a replaceable cartridge.

8. The device of claim 7, wherein said replaceable cartridge is disposable.

9. The device of claim 7 or 8, wherein said replaceable cartridge is sealed.

10. The device of any one of claims 7 to 9, wherein said replaceable cartridge is connected to said carousel via a canister mount.

11. The device of any one of claims 7 to 10, wherein said replaceable cartridge further comprises a battery.

12. The device of any of the preceding claims, wherein said device further comprises a drive motor and gear box component configured to drive said blade.

13. The device of claim 12, wherein said blade is configured to move in a reciprocating manner.

14. The device of claim 12 or 13, wherein said drive motor is configured to start moving said blade when a needle contacts said destruction module and stop moving when said needle is destroyed.

15. The device of any of the preceding claims, wherein said device further comprises an internal power source.

16. The device of any of the preceding claims, wherein said device further comprises an indicator that indicates when said disposal canister is full.

17. The device of any of the preceding claims, wherein said needle is a hypodermic needle or a suture needle.

18. The device of any of the preceding claims, wherein said needle is attached to a syringe.

19. The device of any of the preceding claims, wherein said blade comprises an oscillating component and a stationary component.

20. A method of disposing a needle, comprising:
a) inserting said needle in the needle entry port of the device of any one of claims 1 to 19;
b) advancing said needle to said destruction component; and
c) destroying said needle using said destruction component.

21. The method of claim 20, further comprising step of removing said replaceable cartridge and installing a new replaceable cartridge.

22. The method of any one of claims 20 to 21, wherein said device is used in a home, a hospital, a nursing home, a public restroom, a research lab, a hotel, a clinic, or an ambulance.
